(19) United States
(12) Patent Application Publication
(10) Pub. No.: US 2023/0012758 A1
(43) Pub. Date: Jan. 19, 2023

(54) SUBJECT MONITORING

(71) Applicant: Canaria Technologies Pty Ltd, Brisbane City, Queensland (AU)

(72) Inventors: Robert James FINEAN, Brisbane City (AU); Alexandra Sorina MOSS, Brisbane City (AU); Alexander Joshua GILMOUR, Brisbane City (AU); Christopher Graham CONWAY LAMB, Brisbane City (AU)

(21) Appl. No.: 17/780,433

(22) PCT Filed: Oct. 11, 2021
(86) PCT No.: PCT/AU2021/051184

§ 371 Date: May 26, 2022

(30) Foreign Application Priority Data
Oct. 30, 2020 (AU) 2020903944

(51) Int. Cl. A61B 5/026; A61B 5/00; A61B 5/024

(52) U.S. Cl. CPC A61B 5/0261; A61B 5/6816; A61B 5/6838; A61B 5/7264; A61B 5/0022; A61B 5/02416; A61B 2560/0456; A61B 2560/0214; A61B 2503/20

(57) ABSTRACT

A monitoring system for monitoring a biological subject including a monitoring device having a housing configured to be attached to or supported by an ear of the subject in use, one or more sensors, the one or more sensors including a photoplethysmogram (PPG) sensor provided in the housing and configured to measure attributes of blood flow within the ear, and a monitoring device processor configured to acquire sensors signals from the one or more sensors and generate sensor data at least partially in accordance with signals from the one or more sensors. A transmitter is provided that transmit the sensor data with one or more processing systems receiving the sensor data, analyzing the sensor data and generating a health state indicator indicative of a health state of the subject.

### BACKGROUND OF THE INVENTION

The present invention relates to a method and apparatus for monitoring a biological subject, and in one particular example to a method and apparatus for monitoring a biological subject using a monitoring device that monitors blood flow in an ear of the subject.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

It is known to measure or monitor biometric characteristics or vital signs of a person, such as blood oxygen saturation level or pulse rate, to assess the general health of that person, or to identify specific threats, particularly in an industrial workplace, or similar. For example, cognitive fatigue causes two thirds of all heavy industrial accidents in Australia, costing the resources sector up to AU$15 M per incident. Similarly, heat exhaustion is a global problem that costs an average of AU$41 B due to related injuries, rostering expenses, investigations & rehabilitation.

Specifically, in the case of mining, oil and gas in 2019, cognitive fatigue was cited as the underlying cause of 144 fatalities, where 43 (>30%) were machinery operators and drivers. Heat exposure is supervised broadly based on threshold levels, however individuals respond differently to different conditions so implementing a more individualized monitoring system is prudent for companies in the mining, oil and gas industries.

Construction workers are a group that are particularly vulnerable to health risks because they can be exposed to extreme environmental factors daily. Increasing average temperatures—particularly in urban areas where construction is most prevalent, highlight a need for heat exposure monitoring of construction workers. As little as fifteen minutes in the time a break is taken can be the difference between a worker who is forced to take time off for heat exposure or a worker who is more productive.

In aviation, personnel from the ground crew to the control room in the aviation industry are exposed to working conditions where the risk of fatigue is high. Ensuring that the cognitive capabilities of these staff are at optimum levels is essential as it can increase safety and operational efficiency while reducing operating costs.

For logistics, the transportation industry understands dealing with long periods of high level concentration as sole or lone workers, it is a part of their day to day lives. Monitoring fatigue levels and providing personnel with alerts and notifications as to optimal break times will not only reduce the number of incidents but also lead to increased productivity.

Predictive Biometrics Systems in defense are incredibly applicable when it comes to Defense Forces. From optimizing training methods to monitoring soldiers in the field, ensuring that defense personnel are operating at the highest level of readiness is essential for successful operations.

Devices traditionally used for monitoring vital signs in a hospital environment are typically relatively bulky and uncomfortable and therefore may not be suitable for use in other environments, such as industrial workplaces, in space, or similar.

AU2018101872 describes an earpiece mountable behind an ear of a user. The earpiece includes a casing and, disposed within the casing, a power source and circuitry for measuring a biometric characteristic of the user using reflectance pulse oximetry. The power source is arranged to supply power to the circuitry. Further examples relate to a monitoring system including an earpiece mountable behind an ear of a user and a charging apparatus for charging a power source of the earpiece.

### SUMMARY OF THE PRESENT INVENTION

In one broad form, an aspect of the present invention seeks to provide a monitoring system for monitoring a biological subject, the monitoring system including: a monitoring device including: a housing configured to be attached to or supported by an ear of the subject in use; one or more sensors, the one or more sensors including a photoplethysmogram (PPG) sensor provided in the housing and configured to measure attributes of blood flow within the ear; a monitoring device processor configured to: acquire sensors signals from the one or more sensors; and, generate sensor data at least partially in accordance with signals from the one or more sensors; a transmitter configured to transmit the sensor data; and, one or more processing systems configured to: receive the sensor data; analyze the sensor data; and, generate a health state indicator indicative of a health state of the subject.

In one broad form, an aspect of the present invention seeks to provide a method system for monitoring a biological subject including: using a monitoring device including: a housing configured to be attached to or supported by an ear of the subject in use; one or more sensors, the one or more sensors including a photoplethysmogram (PPG) sensor provided in the housing and configured to measure attributes of blood flow within the ear; and, a monitoring device processor to: acquire sensors signals from the one or more sensors; and, generate sensor data at least partially in accordance with signals from the one or more sensors; using a transmitter to transmits the sensor data; and, using one or more processing systems to: receive the sensor data; analyze the sensor data; and, generate a health state indicator indicative of a health state of the subject.

In one broad form, an aspect of the present invention seeks to provide a monitoring system for monitoring a biological subject, the monitoring system including one or more processing systems configured to: receive sensor data from one or more sensors in a monitoring device worn by a subject; analyze the sensor data by: determining one or more features derived from sensor signals; use the features and at least one computational model to determine a health state indicator indicative of a subject health state, the at least one computational model being at least partially indicative of a relationship between different subject health states and one or more features.

In one broad form, an aspect of the present invention seeks to provide a method for monitoring a biological subject, the method including in one or more processing systems: receiving sensor data from one or more sensors in a monitoring device worn by a subject; analyzing the sensor data by: determining one or more features derived from sensor signals; use the features and at least one computational model to determine a health state indicator indicative of a subject health state, the at least one computational model being at least partially indicative of a relationship between different subject health states and one or more features.

In one embodiment the PPG sensor is a transmissive PPG sensor.

In one embodiment the PPG sensor includes: at least one radiation source provided in the housing so as to expose the ear lobe to electromagnetic radiation; and, at least one radiation sensor provided in the housing so as to receive electromagnetic radiation at least one of transmitted through the ear lobe.

In one embodiment the housing includes: an elongate curved main body configured to sit behind a helix of the ear; and, an ear lobe clamp extending from a lower end of the main body, the ear lobe clamp being configured to receive an ear lobe of the ear so that the ear lobe is positioned between the main body and the ear lobe clamp.

In one embodiment the ear lobe clamp is rotatably mounted to the main body to allow the monitoring device to be worn on a left or right ear.

In one embodiment at least one radiation source is provided in the ear lobe clamp and wherein at least one radiation sensor is provided in the main body facing the ear lobe clamp.

In one embodiment the PPG sensor includes a first radiation sensor on a first side of the housing and second radiation sensor on a second side the housing and wherein the monitoring device processor is configured to: monitor signals from the first and second radiation sensors; determine an active radiation sensor based on the monitored signals; and, generate sensor data using signals from the active radiation sensor.

In one embodiment the housing includes a securing mechanism to secure the housing the ear, the securing mechanism including at least one of: a hook configured to extend over the ear; and, a clip configured to engage an antihelix of the ear.

In one embodiment: the hook is removably mounted proximate an upper end of the housing; and, the clip is removably mounted to either side of the main body to allow the monitoring device to be secured to a left or right ear.

In one embodiment the one or more sensors include at least one of: an ambient temperature sensor; a skin temperature sensor; a pressure sensor; a humidity sensor; a movement sensor; an accelerometer; a gyroscope; an optical sensor; and, a user input button.

In one embodiment the sensor data is indicative of: an amount of red light transmitted through the skin; an amount of green light transmitted through the skin; an amount of infrared light transmitted through the skin; an amount of light absorbed by the skin; an ambient temperature; a barometric pressure; a relative humidity; a wet bulb temperature; a skin temperature; accelerometer readings; and, gyroscope readings; and, user inputs.

In one embodiment the monitoring device includes at least one of: a haptic motor; an optical indicator; and, a speaker.

In one embodiment the sensor data includes at least one of: raw sensor signals; and, one or more features derived from raw sensor signals.

In one embodiment the monitoring device at least partially processes the sensor signals by at least one: filtering; amplifying; digitizing; and, parameterizing.

In one embodiment the system includes a dock configured to: charge a power supply in the monitoring device; and, retrieve sensor data stored in the monitoring device, the retrieved sensor data being transferred to one or more processing systems for analysis.

In one embodiment the one or more processing systems include a client device configured to: analyze the sensor data; generate a health state indicator indicative of a health state of the subject; and, at least one of: generate an alert; output an indication of the health state indicator; and, cause the monitoring device to generate an alert.

In one embodiment the client device is configured to transfer subject data to a processing system, the subject data being indicative of at least one of: sensor data; a health state indicator; and, user input provided in response to output of the health state indicator.

In one embodiment the one or more processing systems include a client device configured to: receive the sensor data; transfer subject data to a processing system, the subject data being at least partially indicative of the sensor data; receive an indication of the health state indicator from the processing system; and, output the health state indicator.

In one embodiment the one or more processing systems are configured to: determine one or more features derived from sensor signals; use the features to determine a health state indicator indicative of a subject health state.

In one embodiment the one or more processing systems are configured to compare one or more feature values to corresponding reference feature values; and, determine the health state in accordance with results of the comparison.

In one embodiment the one or more reference feature values are at least one of: baseline feature values for the subject; previous feature values for the subject; feature values derived from reference subjects having a known health state; and, threshold values.

In one embodiment the one or more features include at least one of: values of raw sensor signals; a pulse feature; a mean heart rate; a heart rate variability feature; a breathing rate; a mean breathing rate; a mean interbeat interval of the heart rate; a median interbeat interval of the heart rate; a standard deviation of the interbeat interval of the heart rate; a median absolute deviation of the interbeat interval; a standard deviation of the difference in the interbeat interval; a median absolute deviation of the difference in the interbeat interval; a percentage difference in Interbeat interval >50 ms; a percentage difference in Interbeat interval >20 ms; a square root of the mean of the successive differences between heart rates; an area under the curve of the heart rate wave; an energy of the power of the Heart Rate Variability (HRV) signal; a proportion of the HRV energy in the Low Frequency band; a proportion of the HRV energy in the High Frequency band; a ratio between HRV signal within the low and high frequency bands; an entropy of the HRV signal; an entropy of the PPG signal; a positive/negative ratio of the Systolic wave; a ratio of the positive Systolic and Diastolic waves; a maximum slope of the Systolic wave; a time to peak of the Systolic wave; an energy of the PPG signal in volts; a proportion of the PPG energy in a Very Low Frequency band; a proportion of the PPG energy in a Low Frequency band; a proportion of the PPG energy in a Medium Frequency band; a proportion of the PPG energy in a High Frequency band; a ratio between the proportion of the PPG energy in the Low Frequency band and proportion of the PPG energy in the high Frequency band; a saturation of Peripheral Oxygen in the blood; a median change in accelerometer signals; a 90th quantile of the accelerometer changes; a 95th quantile of the accelerometer changes; a 99th quantile of the accelerometer changes; a maximum accelerometer change; a median change in the gyroscope signals; a 90th quantile of the gyroscope changes; a 95th quantile of the gyroscope changes; a 99th quantile of the gyroscope changes; a maximum gyroscope change; a power spectral density of Interbeat intervals; a power spectral density of Interbeat intervals in a frequency band 0.04 Hz to 0.15 Hz; a power spectral density of Interbeat intervals in a frequency band 0.16 Hz to 0.5 Hz; a ratio of power spectral density of Interbeat intervals in different frequency bands; an integral of a power spectral density of a signal; an integral of a power spectral density of a signal in a frequency band 0 Hz to 0.3 Hz; an integral of a power spectral density of a signal in a frequency band 1.2 Hz to 1.9 Hz; a mean ambient temperature; an ambient temperature range; a mean wet temperature; a wet temperature range; a wet temperature standard deviation; a mean skin temperature; a skin temperature standard deviation; a skin temperature range; a mean ambient relative humidity; an ambient relative humidity standard deviation; an ambient relative humidity range; a mean ambient pressure; an ambient pressure standard deviation; an ambient pressure range; a PPG vector saturation; a PPG vector noise; a PPG vector noise scale; and, a PPG vector signal variance.

In one embodiment the one or more processing systems determine at least one of: at least two features; at least three features; at least four features; at least five features; at least six features; at least seven features; at least eight features; at least nine features; and, at least ten features.

In one embodiment the wherein the one or more processing systems are configured to use the one or more features and a computational model to determine the health state, the at least one computational model being at least partially indicative of a relationship between different subject health states and one or more features.

In one embodiment the at least one computational model is obtained at least in part by: applying machine learning to reference features derived from one or more reference subjects having known health states; and, applying machine learning to features derived from the subject.

In one embodiment the at least one computational model is obtained at least in part by: developing a generic model by applying machine learning to reference features derived from one or more reference subjects having known health states; and, modifying a generic model to create a subject specific model by applying machine learning to features derived from the subject.

In one embodiment the at least one computational model includes one or more respective computational models for each of a plurality of health states.

In one embodiment the at least one computational model includes at least one of: boosted classifiers that classify aggregated time segments into categories relating to at least one health state; a rolling auto-regressive integrated moving average model applied to key features and raw data to predict risk of at least one health state; and, a long short-term memory model using a recursive deep learning approach which utilizes a previous hours' worth of data to predict risk of at least one health state.

In one embodiment the health state indicator is indicative of one or more of a plurality of health states, including at least one of: cognitive fatigue; heat stress; a risk of cognitive fatigue; a risk of heat stress; and, collapse or non-responsiveness.

In one embodiment a risk of cognitive fatigue is determined using at least one of: a low frequency band of the heart rate signal; a high frequency band of the heart rate signal; a power of the heart rate signal; a barometric pressure; a humidity; an ambient temperature; a PPG signal; an entropy of the PPG signal; a mean interbeat interval of the heart rate; a median interbeat interval of the heart rate.

In one embodiment a risk of heat stress is determined using at least one of: raw PPG signals; a skin temperature; a wet bulb temperature; an ambient temperature; a relative humidity; a barometric pressure; an entropy of a heart rate variability signal; a square root of the mean of the successive differences between heart rates; a mean interbeat interval of the heart rate; a median interbeat interval of the heart rate; UV exposure levels; and, an area under the curve of the Heart Rate wave.

In one embodiment a collapse or non-responsiveness is determined using at least one of: a heart rate; a change in heart rate; a PPG signal; a change in blood oxygenation; accelerometer readings; gyroscope readings; a median change in accelerometer signals; a 90th quantile of the accelerometer changes; a 95th quantile of the accelerometer changes; a 99th quantile of the accelerometer changes; a maximum accelerometer change; a median change in the gyroscope signals; a 90th quantile of the gyroscope changes; a 95th quantile of the gyroscope changes; a 99th quantile of the gyroscope changes; and, a maximum gyroscope change.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:

FIG. 1A is a schematic diagram of an example of a system for monitoring a subject;

FIG. 1B is a schematic side view of an example of the system of FIG. 1A in use;

FIG. 2 is a flow chart of an example of a process for monitoring a subject using the system of FIG. 1A;

FIG. 3 is as schematic diagram of a distributed architecture for monitoring a subject;

FIG. 4 is as schematic diagram of an example of a processing system of FIG. 3;

FIG. 5 is a schematic diagram of an example of a client device of FIG. 3;

FIG. 6A is a schematic right side view of an example of a monitoring device for monitoring a subject;

FIG. 6B is a schematic left side view of the monitoring device of FIG. 6A;

FIG. 6C is a schematic rear side perspective view of the monitoring device of FIG. 6A;

FIG. 6D is a schematic rear view of the monitoring device of FIG. 6A;

FIG. 6E is a schematic plan view of the monitoring device of FIG. 6A;

FIG. 6F is a schematic underside view of the monitoring device of FIG. 6A;

FIG. 6G is a schematic front left underside perspective view of the monitoring device of FIG. 6A, with an earlobe clamp and a clip removed;

FIG. 6H is a schematic front left side perspective view of the monitoring device of FIG. 6A, with part of the housing removed;

FIG. 6I is a schematic front right side perspective view of the monitoring device of FIG. 6A, with part of the housing removed;

FIG. 6J is a schematic perspective front view of an example of a dock for a plurality of monitoring devices;

FIG. 6K is a schematic front view of an example of a docking module of the dock of FIG. 6J;

FIG. 6L is a schematic rear view of the docking module of FIG. 6K;

FIG. 6M is a schematic perspective front view of the docking module of FIG. 6K;

FIG. 6N is a schematic perspective rear view of the docking module of FIG. 6K;

FIGS. 7A and 7B are a flow chart of an example of a process for monitoring a subject;

FIG. 8 is a graph of an example of a PPG signal for a single cardiac cycle; and,

FIG. 9 is a flow chart of an example of a process for generating a computational model.

### DETAILED DESCRIPTION OF THE PREFERRED EMBODIMENTS

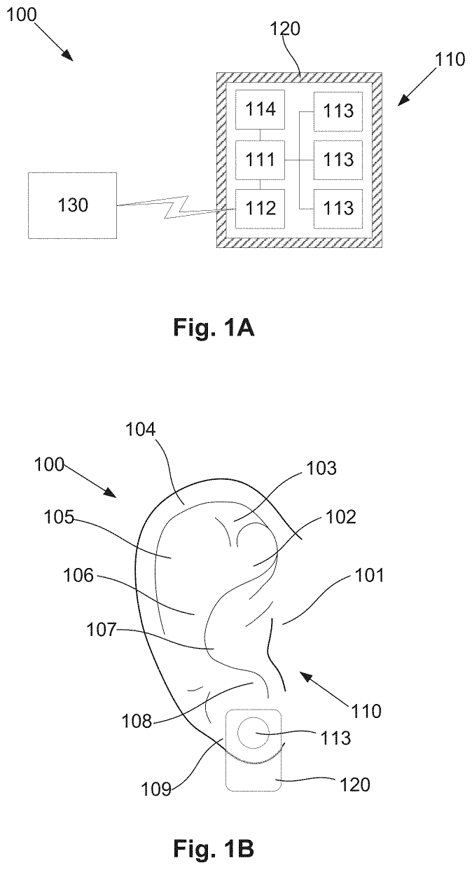

An example of a system for monitoring a biological subject will now be described with reference to FIGS. 1A and 1B.

In this example, the system 100 includes a housing 120 configured to be attached to or supported by an ear of the subject, which typically includes a tragus 101, an inferior crus 102, a superior crus 103, a helix 104, a scapha 105, an antihelix 106, a concha 107, an antitragus 108 and a lobule (earlobe) 109. The housing could be supported or attached to the ear in any suitable manner, and a specific example housing will be described in more detail below.

The monitoring device 110 includes one or more sensors 113 provided in the housing, each of which is adapted to sense one or more characteristics of the subject and/or environment. In particular, the one or more sensors typically include at least a photoplethysmogram (PPG) sensor that is configured to measure attributes of blood flow within the ear. However, additional sensors, such as temperature sensors, skin conductance sensors, movement sensors, or the like, could be used, as will be described in more detail below. In the current example, the PPG sensor is configured to sense blood flow within the earlobe 109 of the subject, and in one particular example can be configured to perform this using transmissive PPG sensing. The earlobe is typically used as this is readily accessible, and has good blood flow therethrough, meaning readings performed are an accurate representation of a health state of the subject. Accordingly, the housing 120 is generally configured to at least contact the earlobe, as shown in FIG. 1B, and more typically is positioned on either side of the earlobe to allow electromagnetic radiation to be emitted from a radiation source on one side of the earlobe and transmitted through the earlobe to a sensor on the other side of the earlobe, although other arrangements could be used.

The monitoring device 110 also typically includes a monitoring device processor 111 and a transmitter 112, as well as an optional memory 114 and power supply (not shown), allowing the monitoring device to monitor the sensors and provide sensor data to one or more processing systems 130, for subsequent analysis.

In use, the monitoring device processor 111 executes instructions in the form of applications software stored in memory to allow the required processes, and in particular to receive sensor signals and allow sensor data to be generated. The monitoring device processor 111 could be any form of electronic processing device such as a microprocessor, microchip processor, logic gate configuration, firmware optionally associated with implementing logic such as an FPGA (Field Programmable Gate Array), or any other electronic device, system or arrangement. The monitoring device can use a single processor, or could use multiple processors, with processing performed by one or more of the processors as needed. For the purpose of ease of illustration, the following examples will refer to a single processor, but it will be appreciated that reference to a singular processor should be understood to encompass multiple processor and vice versa, with processing being distributed between the devices as appropriate.

The transmitter 112 could be of any appropriate form, but in one example, is a short range wireless transmitter, such as a Bluetooth system on a chip (SoC), which allows two way communications with the one or more processing systems 130.

The nature of the processing systems 130 will vary depending upon preferred implementation and could include computer systems such as personal computers, laptops, desktop computers, servers, client devices, such as mobile communication devices including smart phones, tablets, or the like. Further examples will be described in more detail below. Again, reference to a processing system should be understood to encompass multiple processing systems and vice versa.

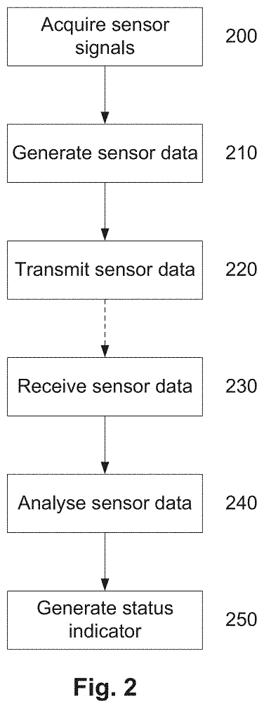

An example of a process for using the system of FIGS. 1A and 1B to monitor a subject will now be described with reference to FIG. 2.

In this example, at step 200 sensor signals are acquired from the one or more sensors 113, including the PPG sensor. At step 210 the monitoring device processor 111 generates sensor data at least partially in accordance with signals from the sensors. The sensor data could be of any appropriate form and may simply be an indication of the sensor signals, or could be information derived therefrom, such as one or more parameters obtained by at least partially analyzing the sensor signals. For example, the monitoring device processor could perform preliminary analysis, such as performing a Fourier transform, convolutions, providing sensor data including frequency domain information instead of raw data. The sensor data may also include other information, such as an identifier used to identify the monitoring device. During this process, the sensor signals acquired by the sensor 113 may also undergo preliminary processing, such as filtering, digitizing, or the like.

At step 220, the sensor data is transmitted by the transmitter 112, allowing this to be received by the processing system(s) 130 at step 230, for example by having the sensor data transferred via a communications network, point-to-point connection, or the like. In one example, this is achieved using a short range wireless communication protocol, such as Bluetooth or similar, although this is not essential and any suitable arrangement could be used.

At step 240 the processing system 130 analyses the sensor data to determine a health state indicator indicative of a health state of the subject. The health state indicator can be of any appropriate form and may include an indication of whether the subject is healthy or is potentially suffering from, or at risk of suffering from, one or more adverse health states. For example, the system could be configured to detect if the subject is undergoing cognitive fatigue or heat stress, or is at risk of undergoing cognitive fatigue or heat stress, or if the subject has collapsed or is non-responsive (referred to as a “man down” scenario).

The health state indicator could be in the form of a numerical value, for example indicating that the user has a 95% chance of being in an adverse health state. However, it will be appreciated that any suitable form of indicator could be used, such as a traffic light indicator to indicate the user's health state is good, intermediate or bad, could be used. The indicator could be associated with a variety of different health states, and might be a composite indicator, including a respective sub-indicator associated with a respective health state. Thus, the indicator could indicate that the user is not suffering from cognitive failure, but has a 90% chance of suffering from heat stroke.

Once a health state indicator subject health state has been determined, this can either be stored for example as part of a profile associated with the respective subject and/or an indication could be presented to the subject. For example, if the one or more processing systems 130 include a client device, the indicator could be could be displayed on a display of the client device, whereas if the processing system is a remote computer system, the indicator could be presented to third party to allow appropriate action to be taken. For example, if the subject is already suffering from cognitive fatigue or heat stress, they might not be in a position to respond appropriately, in which case a supervisor or medical personnel might be alerted.

Additionally and/or alternatively, the indicator could be in the form of an alert. For example, if the subject is at risk of cognitive fatigue or heat stress, the subject could be alerted, allowing the subject to take corrective action, such as taking a break from work. The alert could be generated by the processing system 130, but may also be generated by the monitoring device 110, for example having the monitoring device generate an audible tone, or provide haptic feedback. Even if the subject is currently deemed healthy, information could be displayed, such as presenting a numerical indicator representative of a heat stress risk, allowing the subject to monitor their own condition, and hence avoid cognitive fatigue or heat stress.

Accordingly, it will be appreciated that the above described system enables monitoring of a subject to be performed. In particular, the above described system allows the subject to be monitored by using a monitoring device that is supported by or attached to the ear. The monitoring device can also incorporate multiple sensors allowing a range of different parameters to be monitored, in turn providing a range of useful insights into how the subject is currently functioning.

Sensor data can be transferred to one or more processing systems, such as a subject's smart phone, or remote servers, allowing the data to be analyzed to ensure the subject is in a healthy health state. Performing the analysis on a separate processing system reduces the level of computation required to be performed by the monitoring device, allowing this to be implemented using relatively cheap and straightforward sensors and associated hardware, and reducing power requirements. This, in turn, makes this suitably sized and sufficiently lightweight to allow the monitoring device to be worn for prolonged periods of time, whilst remaining unobtrusive during day to day activities. Nevertheless, the subject can be alerted to any issues using the monitoring device itself, and/or using a local processing system, such as a smart phone, whilst additionally and/or alternatively allowing remote monitoring by supervisors, medical personnel, or other responsible parties.

Additionally, this arrangement also allows data to be readily stored and analyzed centrally. This in turn allows the data to be used in understanding what particular circumstances or parameters lead to, or are likely to lead to, certain health states arising. In one example, this is performed using machine learning to generate computational models, which can then be used in generating the health state indicators. It will be appreciated that collecting additional data allows models to be refined and hence improved, thereby improving the effectiveness of the system at identifying different health states. However, this is not essential and alternatively and/or additionally, analysis can be performed by comparison of signals or derived features to reference feature values, such as previous measurements for the subject, or other reference subjects having known health states. In another example, the reference feature values could be threshold values, for example based on international standards, such as ISO standards, and/or occupational health and safety laws. In this example, the threshold values represent limits of safe heat exposure levels, with results of the comparison being used to determine if these safe levels have been exceeded.

In one particular example, the above described system measures raw PPG wavelengths, skin temperature, movement and environmental data. From these primary sensor readings, a number of metrics and/or features are derived, such as blood oxygen saturation, breathing rate and heart rate variability. Combinations of these metrics or features can be used, optionally with machine and deep learning AI models, to generate health state indicators and warning alerts where needed.

A number of further features will now be described.

As mentioned above, the system typically includes a PPG sensor, and in one preferred example, the system utilizes transmissive PPG to measure blood flow changes in the earlobe. The approach uses at least one radiation source provided in the housing so as to expose the earlobe to electromagnetic radiation and at least one radiation sensor provided in the housing so as to receive electromagnetic radiation transmitted through the earlobe. Thus, this approach applies multiple frequencies of electromagnetic radiation to the subject, typically including electromagnetic radiation having wavelengths in the Infrared, Red and Green regions of the spectrum, with this radiation being transmitted through the skin to a sensor on the other side of the earlobe. The sensor measures changes in the amount of light absorbed to illustrate activity within the subject's circulatory system. This arrangement results in minimal light interference, which in turn enables significantly reduced inaccuracy and increases the fidelity in the data collected because different light spectrums are absorbed by the skin in different ways.

It should be noted that this contrasts with more traditional implementations in most common wearable devices where reflectance PPG is used. In this example, green light is shone into a user's skin and then the amount reflected back is recorded. However this approach is susceptible to inaccuracies due to:
Melanin (responsible for skin pigmentation) in a person's skin is a very good absorber of green light, which means darker, tattooed, freckled or skin affected by any other normal physiologic variation will produce inaccurate readings.
Increased light exposure due to movement of the user's arm.
Movement of the device on the wrist affecting the amount of light absorbed by the photo sensors.

In the current arrangement, the sensors can read the amount of blood in the underlying tissue 100 times a second, which is sufficient to illustrate different phases of heart function. Paired with the accuracy provided with the transmissive approach, this level of detail enables high level processing of the raw signal to produce well known measures within the QRS Complex and other meaningful information about a user's wellbeing, including, but not limited to features such as:
Heart Rate
Heart Rate Variability
Breathing Rate
Interbeat Interval
Predictability of the PPG
Ratios highlighting Systolic and Diastolic waves
Power of the circulatory system

In one example, the housing includes an elongate curved main body configured to sit behind a helix of the ear and an earlobe clamp extending from a lower end of the main body, the earlobe clamp being configured to receive an earlobe of the ear so that the earlobe is positioned between the main body and the earlobe clamp. This secures the housing relative to the earlobe, reducing relative movement between the housing and earlobe, whilst also allowing the radiation source and sensor to be positioned on either side of the earlobe, which in turn allows the PPG sensor to make reliable readings. Additionally, the configuration of the elongate curved main body, allows this to sit unobtrusively behind the ear, whilst providing sufficient physical size to incorporate the sensors and associated electronic components, allowing this to be used for prolonged time periods.

In one example, the earlobe clamp is rotatably mounted to the main body to allow the monitoring device to be worn on a left or right ear. In this arrangement, radiation source(s) can be provided in the earlobe clamp and radiation sensor(s) in the main body facing the earlobe clamp. To allow for the rotation of the clamp, the PPG sensor can include a first radiation sensor on a first side of the housing and second radiation sensor on a second side the housing, so that transmitted radiation can be detected by either the first or second sensors, depending on the orientation of the earlobe clamp. It will also be appreciated that reverse arrangements could be used, with sensors in the earlobe clamp and radiation sources in the main body.

In this example, the monitoring device processor 111 can be configured to monitor signals from the first and second radiation sensors and use this to determine an active radiation sensor based on the monitored signals. For example, by analyzing the spectra of any detected radiation, the processor can identify which of the first or second sensors are receiving radiation emitted by the radiation source(s), with the processor generating sensor data using signals from these active radiation sensor(s). Thus, it will be appreciated that the profile of received radiation that has been transmitted through the earlobe will differ significantly compared to ambient radiation, and this can be easily used to identify the orientation of the earlobe clamp, and hence ensure measurements performed are using the correct sensor and are accurate.

The housing can include a securing mechanism to secure the housing to the ear. The securing mechanism could include a hook configured to extend over the ear and/or a clip configured to engage an antihelix of the ear. In this latter case, the clip can be removably mounted to either side of the main body to allow the monitoring device to be secured to a left or right ear, whilst the hook can be removably mounted proximate an upper end of the housing. These arrangements are particularly suitable as they are unobtrusive and do not obstruct the ear canal, allowing the system to be used without interfering with the subject's hearing. This allows the monitoring device to be used with headphones or headsets, as well protective equipment, such as ear plugs, ear defenders, or the like, as required. The clip and hook are also removable and adjustable, allowing the clip and hook to be configured to ensure the monitoring device can be adequately secured to the ear, whilst remaining comfortable.

The monitoring device can include a range of different sensors including, but not limited to an ambient temperature sensor, a skin temperature sensor, a skin conductance sensor, a pressure sensor, a humidity sensor, a movement sensor, such as an accelerometer and/or gyroscope, a microphone, an optical sensor, a user input button, or the like. It will be appreciated from this that the sensor data could indicative of a range of different metrics, including any one or more of an amount of red light transmitted through the skin, an amount of green light transmitted through the skin, an amount of infrared light transmitted through the skin, an amount of light absorbed by the skin, an ambient temperature, a barometric pressure, a relative humidity, a wet bulb temperature, a skin temperature, a galvanic skin response, accelerometer readings, gyroscope readings and/or user inputs. The monitoring device may also include a haptic motor, an optical indicator, such as an LED, and/or speaker to allow alerts or other feedback to be provided to the subject.

The sensor data can include signals from the sensor(s), in other words raw sensor data, or might include partially processed data. For example, this could include sensor signals that have been at least partially processed by filtering, amplifying, digitizing or parameterizing. Additionally and/or alternatively, the sensor data could include one or more features derived from the sensor signals, such as heart rate indications, heart variability measurements, or similar, as will be described in more detail below. In this regard, it will be appreciated that the sensor data transmitted may vary depending on the circumstances. For example, having access to the raw data can be useful for the purpose of training models, but may not be required for real time monitoring. Additionally, the raw sensor data could be large in size and hence not suited for transfer in real time.

Accordingly, in one example, the monitoring device is configured to generate and transmit sensor data indicative of metrics or features derived from the sensor data whilst in use. This reduces the bandwidth required to upload data while in use, but still allows real-time monitoring to be performed. During this process, the monitoring device stores raw sensor data, and then uploads this to a remote processing system at a later time, for example, when the monitoring device is undergoing charging or similar. In this regard, the monitoring device could be coupled to a dock configured to charge a power supply, such as a battery, in the monitoring device. It will be appreciated that this could be achieved using inductive or connective/conductive based charging, depending on the preferred implementation. Additionally, the dock can operate to download the raw sensor data from the monitoring device and upload this to one or more processing systems, such as a server or other similar system, for analysis. This allows for a subsequent more in-depth analysis of the raw data.

In one example, the one or more processing systems include a client device, such as a mobile phone or tablet, which is configured to analyze the sensor data and generate the health state indicator. The client device can then output the health state indicator, for example presenting this on a display. Additionally and/or alternatively, the client device could generate an alert, such as an audible or haptic notification, alerting the subject to an adverse or a risk of an adverse health state. Similarly, the client device could communicate with the monitoring device and cause the monitoring device to generate an alert, such as an audible or haptic alert, warning the subject of the adverse or potentially adverse health state. Thus, this allows analysis to be performed and results output locally, substantially in real time. This is useful for alerting the subject in the event that there is a risk of them developing an adverse health state, such as heat exhaustion or similar.

In this example, the client device can also be configured to transfer subject data to a remote processing system, such as a cloud based server, or similar, with the subject data including sensor data, a health state indicator and optionally any user input provided in response to output of the health state indicator. Thus, in this example, analysis is performed by the client device, with results of the analysis and any resulting action taken being uploaded for logging purposes for example to serve as a record of what information was presented to the subject, and what resulting action, if any, was taken. This can also be used to alert third parties, such as supervisors or medical personnel, allowing interventions to be performed as needed.

In another example, the client device can be configured to receive the sensor data, transfer subject data to a processing system, the subject data being at least partially indicative of the sensor data, receive an indication of the health state indicator from the processing system and then output the health state indicator. Thus, in this example, the client device does not perform analysis, but rather uploads the data to a remote processing system, such as a server for analysis, receiving the resulting indicator and allowing this to be presented to the user. This can be useful in the event that the degree of computation required to be performed by the client device is prohibitive. In this instance, the client device relays information derived from the sensor data to the server, allowing for the analysis to be performed, whilst still allowing the monitoring device to use a lower energy wireless communications link with the client device, to avoid undue battery drain associated with long range wireless communications links.

Analysis of the sensor signals is typically achieved by comparing features derived from the sensor signals to equivalent features measured for subjects having known health states. Thus, this approach allows sensor signals from individuals having a known health state (hereinafter referred to as reference subjects for ease of illustration) to be used as a baseline for comparison. The system can then analyze current sensor readings from a subject and use this to determine if the subject has a healthy or potentially adverse health state.

In one example, the one or more processing systems are configured to determine one or more features derived from signals from the one or more sensors. This could involve analyzing the signals directly, or receiving features that have been derived onboard the monitoring device. The features could also include raw signal values. The processing system then uses the features to determine a health state indicator indicative of a subject health state.

In one example, the health state is determined by comparing one or more feature values to corresponding reference feature values, with the health state being determined in accordance with results of the comparison. The reference feature values could be of any appropriate form and could for example include baseline feature values for the subject and/or previous feature values for the subject. For example, a subject could be monitored over a period of time to establish “normal” values and/or ranges of values for features, including raw sensor signals or other features derived therefrom. In this instance, the comparison can be used to identify if the subject is within or has deviated from these “normal” values or ranges, which in turn can be used to determine a health state. In this regard, the comparison can assess not only that the features differ, but can also look at the magnitude and/or direction of a difference, for example making a different assessment of health state depending on whether a feature such as heart rate variability as increased or decreased relative to normal values.

The reference feature values could additionally and/or alternatively be feature values derived from reference subjects having a known health state. This allows measurements from multiple different subjects to be used to establish ranges of feature values that are indicative of particular health states. In this example, the reference feature values could be obtained from reference subjects having similar physical characteristics to the subject. Thus, it will be appreciated that the a 20 year old male subject may react differently to heat stress to an 80 year old female subject. Accordingly, comparison to reference feature values established for reference subjects having similar physical characteristics to the subject can help ensure accuracy of any analysis.

In one example, a range of different features can be analyzed, including one or more of: raw sensor signals; a pulse feature; a heart rate; a mean heart rate; a heart rate variability feature; a breathing rate; a mean breathing rate; an interbeat interval of the heart rate; a mean interbeat interval of the heart rate; a median interbeat interval of the heart rate; a standard deviation of the interbeat interval of the heart rate; a median absolute deviation of the interbeat interval; a standard deviation of the difference in the interbeat interval; a median absolute deviation of the difference in the interbeat interval; a percentage difference in Interbeat interval >50 ms; a percentage difference in Interbeat interval >20 ms; a square root of the mean of the successive differences between heart rates; an area under the curve of the heart rate wave; an energy of the power of the Heart Rate Variability (HRV) signal; a proportion of the HRV energy in the Low Frequency band; a proportion of the HRV energy in the High Frequency band; a ratio between HRV signal within the low and high frequency bands; an entropy of the HRV signal; an entropy of the PPG signal; a positive/negative ratio of the Systolic wave; a ratio of the positive Systolic and Diastolic waves; a maximum slope of the Systolic wave; a time to peak of the Systolic wave; an energy of the PPG signal in volts; a proportion of the PPG energy in a Very Low Frequency band; a proportion of the PPG energy in a Low Frequency band; a proportion of the PPG energy in a Medium Frequency band; a proportion of the PPG energy in a High Frequency band; a ratio between the proportion of the PPG energy in the Low Frequency band and proportion of the PPG energy in the high Frequency band; a saturation of Peripheral Oxygen in the blood; a median change in accelerometer signals; a 90th quantile of the accelerometer changes; a 95th quantile of the accelerometer changes; a 99th quantile of the accelerometer changes; a maximum accelerometer change; a median change in the gyroscope signals; a 90th quantile of the gyroscope changes; a 95th quantile of the gyroscope changes; a 99th quantile of the gyroscope changes; a maximum gyroscope change; a power spectral density of Interbeat intervals; a power spectral density of Interbeat intervals in a frequency band 0.04 Hz to 0.15 Hz; a power spectral density of Interbeat intervals in a frequency band 0.16 Hz to 0.5 Hz; a ratio of power spectral density of Interbeat intervals in different frequency bands; an integral of a power spectral density of a signal; an integral of a power spectral density of a signal in a frequency band 0 Hz to 0.3 Hz; an integral of a power spectral density of a signal in a frequency band 1.2 Hz to 1.9 Hz; a mean ambient temperature; an ambient temperature range; a mean wet temperature; a wet temperature range; a wet temperature standard deviation; a mean skin temperature; a skin temperature standard deviation; a skin temperature range; a mean ambient relative humidity; an ambient relative humidity standard deviation; an ambient relative humidity range; a mean ambient pressure; an ambient pressure standard deviation; an ambient pressure range; a PPG vector saturation; a PPG vector noise; a PPG vector noise scale; and, a PPG vector signal variance.

The number and combination of features used will depend on the outcome of training and in particular a variety of different features will generally be trialed, with the resulting model being tested against training data to ascertain a discriminatory power. In general, the number of features used will be at least two features; at least three features; at least four features; at least five features; at least six features; at least seven features; at least eight features; at least nine features; and, optionally at least ten features.

In another example, the health state is determined using at least one computational model. In this regard the computational model is typically at least partially indicative of a relationship between different subject health states and one or more features. Thus, in this example, the computational model is used to allow the particular combinations of features to be related to different health states, so that this can then be used in analyzing the sensor signals.

Whilst the computational model(s) could be derived in any suitable manner, in one example the computational model is trained via machine learning using data collected from the subject and/or one or more reference subjects. Thus, in one example the computational model is obtained by applying machine learning to reference features derived from one or more reference subjects having known health states and/or applying machine learning to features derived from the subject. In particular, in general, a generic model is developed by applying machine learning to reference features derived from one or more reference subjects having known health states, with this being used as a base model for the subject. This generic model is then used to create a subject specific model by applying machine learning to features derived from the subject, so that the base model is modified as additional data is collected until it is specific for the subject, thereby individualizing the model and making it more accurate for the subject being measured.

It will be appreciated that the analysis process can be aided by feedback from users. For example, if a subject is classified as having cognitive fatigue, but is tested and is not in fact suffering, this information can be fed back to refine the reference features values and/or the machine learning process, allowing the system to reclassify behaviours and more accurately identify issues moving forward. In one example, the subject can be asked to provide responses via an input on the monitoring device, with these responses being used to further assess the subject's health state. For example, the subject could be required to respond within a set time limit of a prompt, with the ability of the subject to do this being used as input to assess the subject's health state.

The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail.

As mentioned above, the system can be configured to monitor for a number of different health states, including but not limited to a subject undergoing cognitive fatigue or heat stress, a risk of cognitive fatigue or heat stress, or collapse or non-responsiveness.

In general, one or more different computational models will be used for each health state, as each health state will have a different impact on the different measured features. For example, heart rate variability is one of the most reliable indicators of cognitive fatigue, whilst skin temperature may be more effective in detecting the onset of heat stress.

In one example, an ensemble model is created, which could include boosted classifiers that classify aggregated time segments into categories relating to at least one health state, a rolling auto-regressive integrated moving average model applied to key features and raw data to predict risk of at least one health state, or a long short-term memory model using a recursive deep learning approach which utilizes a previous hours' worth of data to predict risk of at least one health state.

When monitoring a risk of cognitive fatigue, this typically employs one or more of the following features: a low frequency band of the heart rate signal; a high frequency band of the heart rate signal; a power of the heart rate signal; a barometric pressure; a humidity; an ambient temperature; a PPG signal; an entropy of the PPG signal; a mean interbeat interval of the heart rate; or a median interbeat interval of the heart rate.

When monitoring a risk of heat stress, this typically employs one or more of: a skin temperature; a wet bulb temperature; an ambient temperature; a relative humidity; a barometric pressure; an entropy of a heart rate variability signal; a square root of the mean of the successive differences between heart rates; a mean interbeat interval of the heart rate; a median interbeat interval of the heart rate; UV exposure levels, for example determined from meteorological data; or an area under the curve of the Heart Rate wave.

When monitoring collapse or non-responsiveness, this typically employs one or more of a heart rate; a change in heart rate; a PPG signal; a change in blood oxygenation; accelerometer readings; gyroscope readings; a median change in accelerometer signals; a 90th quantile of the accelerometer changes; a 95th quantile of the accelerometer changes; a 99th quantile of the accelerometer changes; a maximum accelerometer change; a median change in the gyroscope signals; a 90th quantile of the gyroscope changes; a 95th quantile of the gyroscope changes; a 99th quantile of the gyroscope changes; or a maximum gyroscope change.

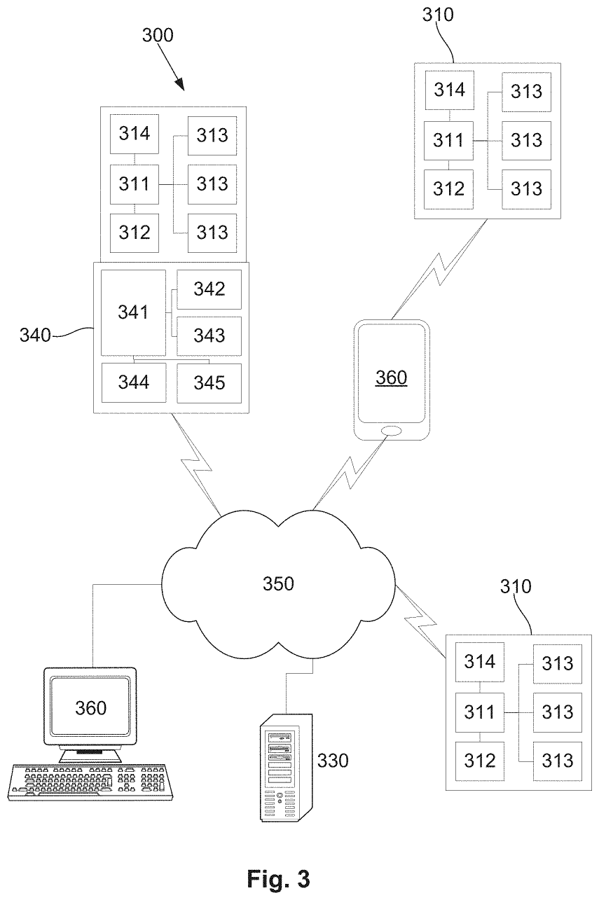

A more specific example will now be described with reference to FIGS. 3 to 6.

In this example, as shown in FIG. 3, the system 300 typically includes a monitoring device 310, broadly similar to the monitoring device 110 described above, and includes a processor 311, transmitter 312, sensors 313 a memory 314, and power supply (not shown). Additionally, a number of processing systems 330 are provided coupled to one or more client devices 360, via the one or more communications networks 350, such as the Internet, and/or a number of local area networks (LANs), or the like.

The system may also include a dock 340, which is adapted to retrieve and upload sensor data from one or more of the monitoring devices 310 to a communications network 350, typically whilst also charging the power supply in the monitoring device 310. The 340 dock can be of any appropriate form, but in one example includes a dock processor 341 and first and second interfaces 342, 343. The dock may also include additional components, such as memory, power supplies or the like, as will be appreciated by persons skilled in the art.

In this example, the first interface 342 is typically adapted to provide short range communications, allowing communication with one or more of the monitoring devices 310, and may include one or more Bluetooth transmitter/receiver chips, physical connections with the monitoring device, or the like. The second interface 343 is a network interface, for providing onward connectivity to one or more communications networks 350.

The dock 340 can also include an input/output device 344, such as a touch screen or similar, allowing a user to interact with the dock and control various functionality, such as retrieval of sensor data from the monitoring devices, and uploading of data. The dock may also include a memory 345 for storing data prior to upload.

In use, the dock processor 341 executes instructions in the form of applications software stored in memory to allow the required processes, and in particular retrieval and upload of sensor data, to be performed. Whilst the dock processor 341 can be a standard microprocessor, such as an Intel Architecture based microprocessor, this is not essential and any suitable arrangement, such as microchip processor, logic gate configuration, firmware optionally associated with implementing logic such as an FPGA (Field Programmable Gate Array), or any other electronic device, system or arrangement, could be used.

Any number of monitoring devices 310, processing systems 330, docks 340 and client devices 360 could be provided, and the current representation being for the purpose of illustration only. The configuration of the networks 350 is also for the purpose of example only, and in practice the processing systems 330 and client devices 360 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, or the like. Thus, the monitoring device 310 could communicate with a client device 360 via a point-to-point connection, or could communicate via the networks 350 or the docks 340.

In this example, the client devices 360 are adapted to analyze sensor data from the monitoring devices 310, and determine a health state indicator, allowing this to be displayed to a subject via the client device 360. The processing systems 330 are generally configured to perform additional analysis, for example to train models, or perform the analysis in the event that this cannot be performed by the client devices. Whilst the processing systems 330 are shown as single entities, it will be appreciated they could include a number of processing systems distributed over a number of geographically separate locations, for example as part of a cloud based environment. Thus, the above described arrangements are not essential and other suitable configurations could be used.

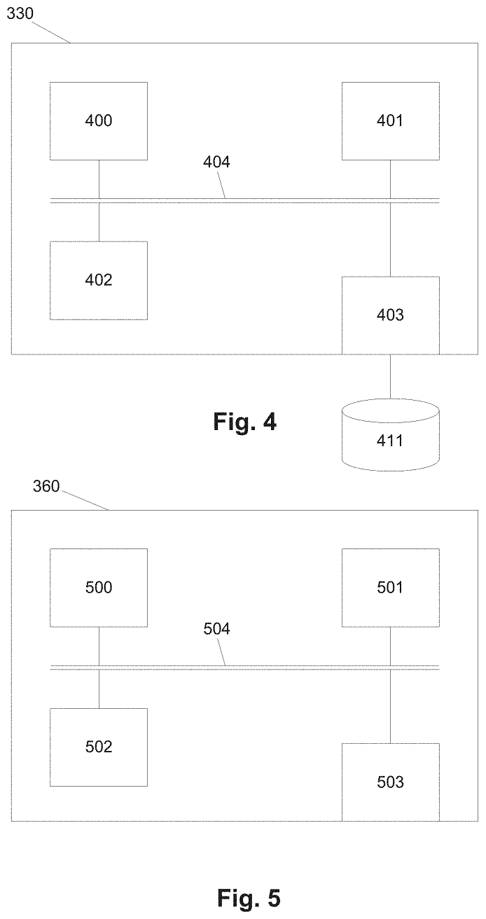

An example of a suitable processing system 330 is shown in FIG. 4. In this example, the processing system 330 includes at least one microprocessor 400, a memory 401, an optional input/output device 402, such as a keyboard and/or display, and an external interface 403, interconnected via a bus 404 as shown. In this example the external interface 403 can be utilized for connecting the processing system 330 to peripheral devices, such as the communications networks 350, databases 411, other storage devices, or the like. Although a single external interface 403 is shown, this is for the purpose of example only, and in practice multiple interfaces using various methods (e.g. Ethernet, serial, USB, wireless or the like) may be provided.

In use, the microprocessor 400 executes instructions in the form of applications software stored in the memory 401 to allow the required processes to be performed. The applications software may include one or more software modules, and may be executed in a suitable execution environment, such as an operating system environment, or the like.

Accordingly, it will be appreciated that the processing system 330 may be formed from any suitable processing system, such as a suitably programmed PC, web server, network server, or the like. In one particular example, the processing system 330 is a standard processing system such as an Intel Architecture based processing system, which executes software applications stored on non-volatile (e.g., hard disk) storage, although this is not essential. However, it will also be understood that the processing system could be any electronic processing device such as a microprocessor, microchip processor, logic gate configuration, firmware optionally associated with implementing logic such as an FPGA (Field Programmable Gate Array), or any other electronic device, system or arrangement.

As shown in FIG. 5, in one example, the client device 360 includes at least one microprocessor 500, a memory 501, an input/output device 502, such as a keyboard and/or display, an external interface 503, and typically a card reader 504, interconnected via a bus 505 as shown. In this example the external interface 503 can be utilized for connecting the client device 360 to peripheral devices, such as the communications networks 350, databases, other storage devices, or the like. Although a single external interface 503 is shown, this is for the purpose of example only, and in practice multiple interfaces using various methods (e.g. Ethernet, serial, USB, wireless or the like) may be provided.

In use, the microprocessor 500 executes instructions in the form of applications software stored in the memory 501, and to allow communication with one of the processing systems 330.

Accordingly, it will be appreciated that the client device 360 be formed from any suitably programmed processing system and could include suitably programmed PCs, Internet terminal, lap-top, or hand-held PC, a tablet, a smart phone, or the like. However, it will also be understood that the client device 360 can be any electronic processing device such as a microprocessor, microchip processor, logic gate configuration, firmware optionally associated with implementing logic such as an FPGA (Field Programmable Gate Array), or any other electronic device, system or arrangement.

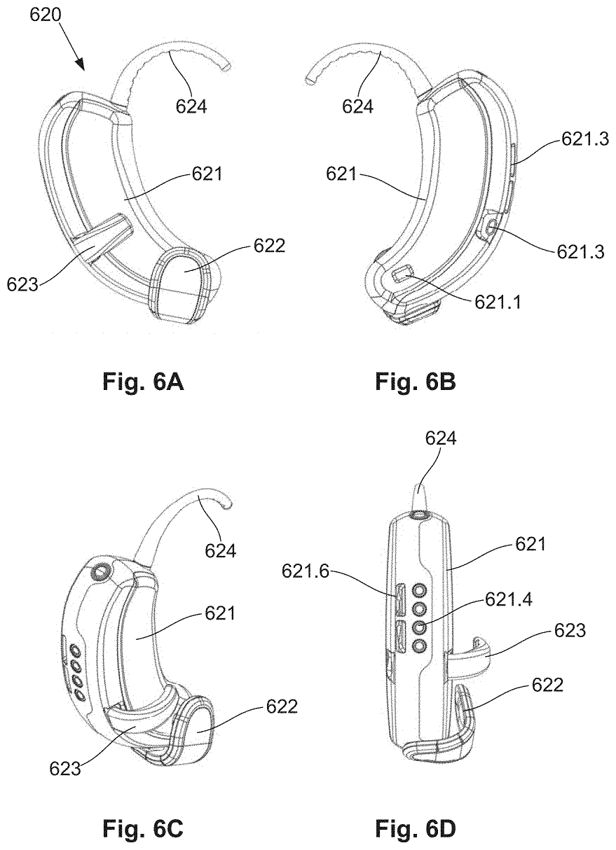

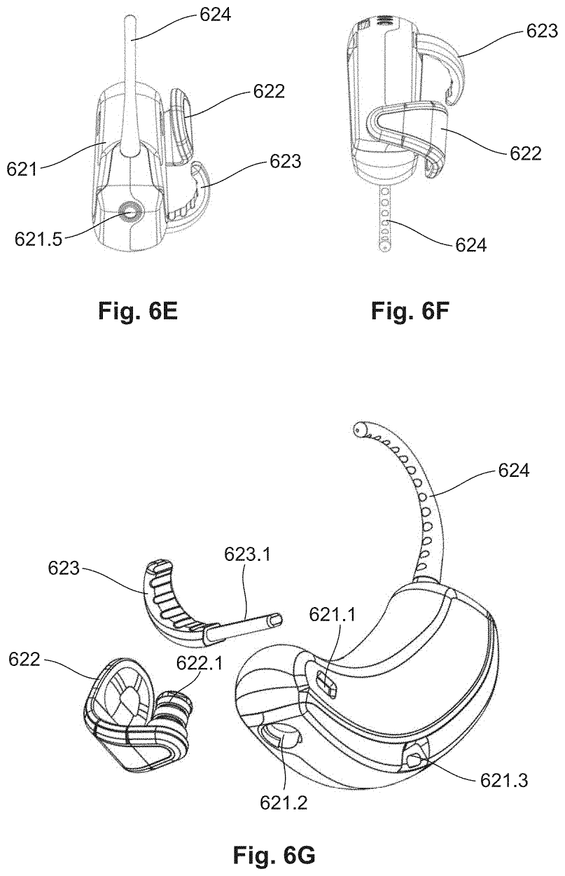

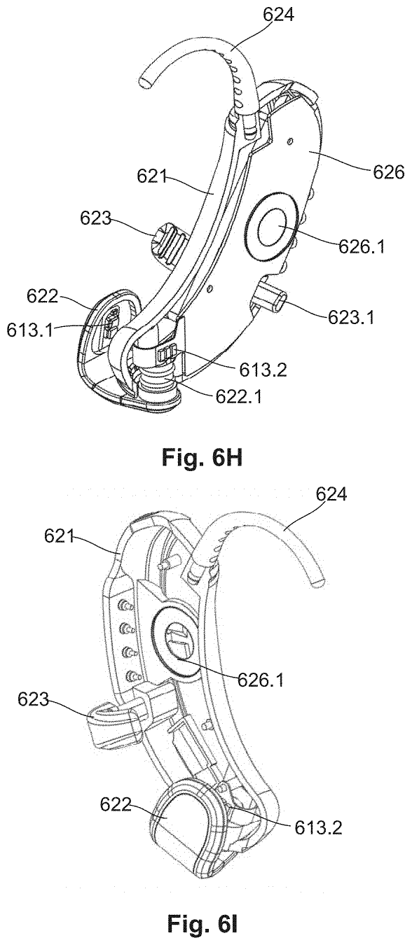

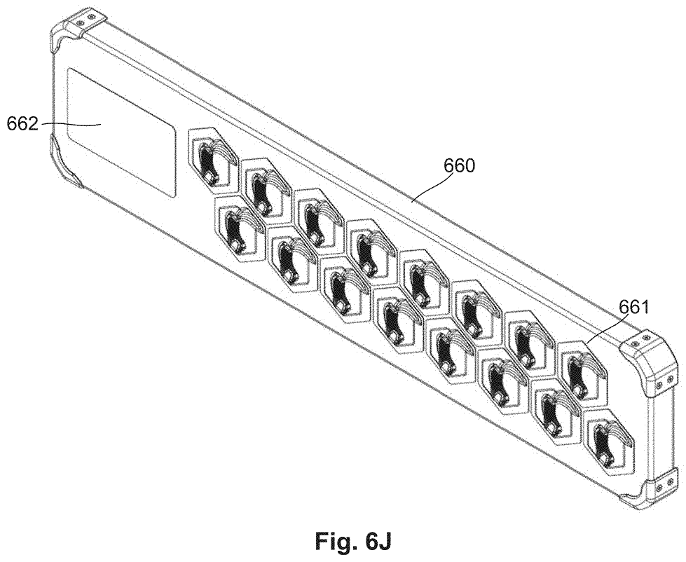

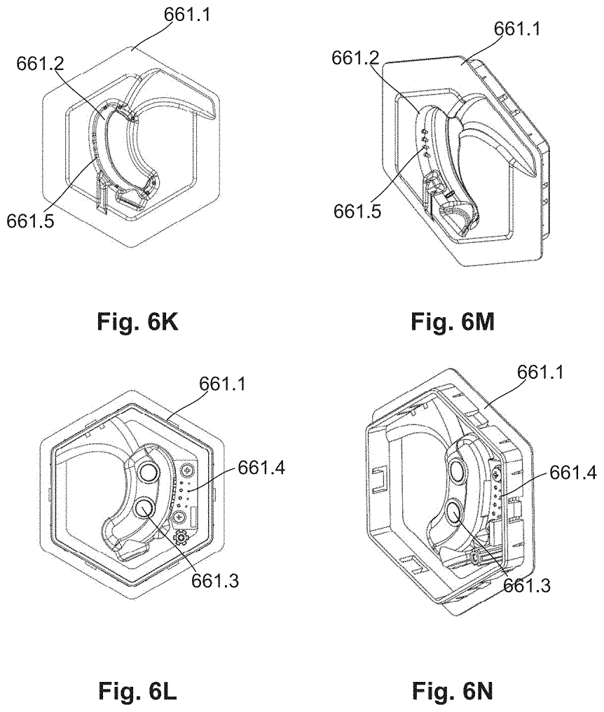

A specific example of the physical configuration of a monitoring device will now be described with reference to FIGS. 6A to 6I.

In this example, the housing 620 includes a main body 621, which is elongate and curved, having a generally rounded crescent shape. The body 621 includes an earlobe clamp 622, which extends laterally from a lower end of the main body 621, before extending upwardly, so that the earlobe clamp extends substantially parallel to a side of the main body 621, allowing an earlobe to be retained between the earlobe clamp 622 and the main body 621. The earlobe clamp 622 includes a shaft 622.1, which in use is located in a socket 621.1 provided in a lower end of the main body 621, allowing the earlobe clamp 622 to be rotatably mounted thereto. This allows earlobe clamp to be moved from a right hand side of the main body 621, shown in FIGS. 6A to 6F, which supports wearing of the monitoring device on the right ear, to the opposite side of the main body 622 (not shown) to support wearing of the monitoring device on the left ear.

The earlobe clamp 622 incorporates radiation sources 613.1, such as laser or light emitting diodes, which emit radiation through a transparent surface of the earlobe clamp 622. The emitted radiation passes through a window 621.1 in the main body, allowing this to be received by the radiation sensors 613.2, such as photodiodes or similar, mounted within the main body 621. Windows 621.1 and radiation sensors 613.2 are mounted on each side of the main body, so that radiation from the radiation sources 613.1 can be received regardless of whether the earlobe clamp is on the left or right side of the main body 621. The radiation sources 613.1 are typically driven via electrical connections, such as wires or the like extending through the shaft 621.1.

The earlobe clamp 622 and/or main body 621 may also include electrodes that contact the ear of the subject, allowing impedance and/or conductance measurements to be performed.

An antihelix clip 623 is provided, which includes a clip shaft 623.1, which in use is mounted in a port 621.3 that extends laterally through the main body 621, allow the clip to be mounted on the left (not shown) or right side (FIGS. 6A to 6F) of the main body 621. The antihelix clip 623 is located towards a rear of the main body 621, mid-way along the body, and is configured to engage the antihelix of the ear in use. The clips are typically flexible and malleable, allowing the subject to adjust the clip configuration to ensure a secure and comfortable fit. The clips are also removable, so that the clips do not need to be used, if not required.

The housing 620 further includes a hook 624 configured to extend over a top of the back of the ear, to thereby support the weight of the monitoring device.

The housing 620 includes a number of electrical connectors 621.4, which can be used for charging an internal battery (not shown) and optionally allowing for data transfer from an onboard memory. Typically this is achieved using the dock 340, which facilities charging and data transfer.

An input button 621.5 may be provided on an upper end of the housing 620. The input button can be used to provide multiple different functions, depending on the situation. For example, this could be used to power the monitoring device on or off, control a pairing mode to allow the monitoring device to be paired with a client device 360 for data transfer. Alternatively, this could be used to allow a subject to provide a response, for example in the event that a response is requested to ensure the subject is responsive. Alternatively, the input button could be replaced by other suitable inputs, such as detecting tapping of the housing, inputs provided via a connected device, such as a smartphone, or the like.

The main body 621 also includes vents 621.6 in a rear face of the main body. The vents are in fluid communication with a Gore membrane 626.1, to allow for pressure equalization for pressure sensing purposes.

It will be appreciated that the housing 620 also typically includes associated circuitry and a battery, which are not shown for clarity. The circuitry would include the monitoring device processor, a memory and wireless transceiver for communication with the client device 360 and/or a communications network 350, depending on the preferred implementation. The circuitry would also include additional sensors, such as the temperature, humidity sensors, or the like, as outlined above.

Specific examples of sensors and components used include PT17-21C/L41/TR8 and VTPS1192HB PPG receivers, XZM2ACR53W-8, XZM2DG53W-8 and XZTNI53W-8 PPG transmitters (actuated by TPS75105DSK LED drivers), an nRF52832-QFAA is the Bluetooth microcontroller, a LSM6DSM accelerometer/gyroscope unit, a BME680 ambient temperature/humidity/barometric pressure sensor, an NCU15XH103F60RC skin temperature sensor, and MT29F4G01ABA or MT29F2G01ABA on-board memory for storing raw data.

An example of a dock for use with multiple monitoring devices will now be described with reference to FIGS. 6J to 6N.

In this example, the dock 340 includes a housing 660 including docking module 661, each of which is configured to receive a respective monitoring device, and a touch screen to allow user interaction with the dock.

As shown in FIGS. 6K to 6N, each docking module includes a body 661.1 that attaches to the housing 660, including a recess 661.2 shaped to receive a monitoring device. A rear surface of the recess includes magnets 661.3 provided on an underside so that the monitoring device magnetically engages with the dock. A printed circuit board 661.4 is provided on a rear side of the body 661.1, adjacent the recess 661.2, with contacts 661.5 extending through a recess side wall, so that the contacts 661.5 engage with electrical connectors 621.4 on the monitoring device, to allow for transfer of power and data.

Example processes of monitoring a subject will now be described in further detail. For the purpose of these examples it is assumed that one or more respective processing systems 330 are servers. In one example, the servers 330 communicate with client devices 360, which transfer data from the monitoring devices 310, allowing this to be analyzed and/or used to generate and/or refine computational models. Similarly, the client devices 360 are typically configured to analyze data, typically using models uploaded from the servers 330, and present results of the analysis to the subject being monitored.

The servers 330 typically execute software, allowing relevant actions to be performed, with actions performed by the server 330 being performed by the processor 400 in accordance with instructions stored as applications software in the memory 401 and/or input commands received from a user via the I/O device 402. It will also be assumed that actions performed by the client devices 360, are performed by the processor 500 in accordance with instructions stored as applications software in the memory 501 and/or input commands received from a user via the I/O device 502.

However, it will be appreciated that the above described configuration assumed for the purpose of the following examples is not essential, and numerous other configurations may be used. It will also be appreciated that the partitioning of functionality between the different processing systems may vary, depending on the particular implementation.

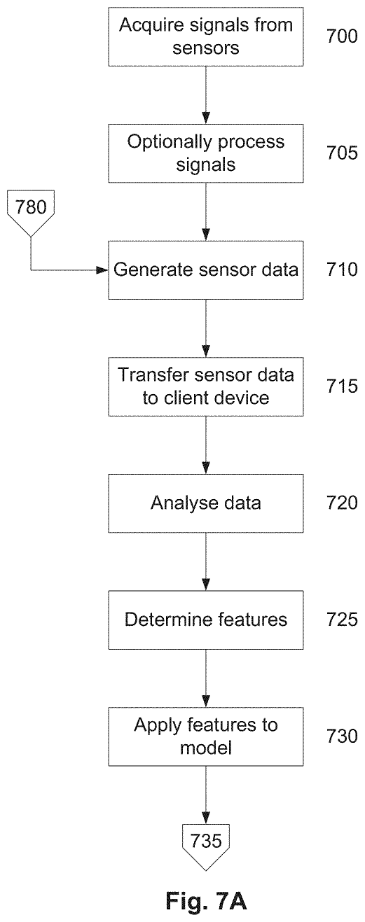

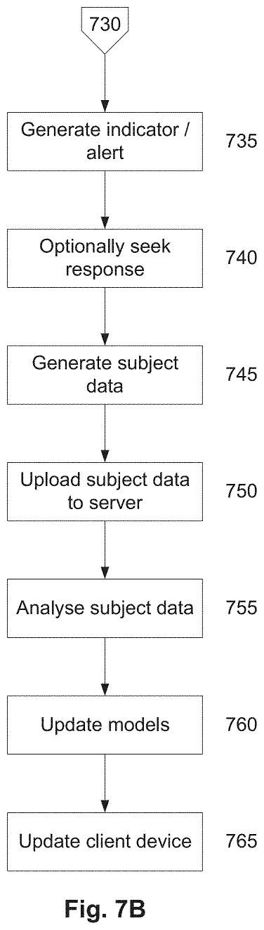

An example of a process for monitoring a subject will now be described with reference to FIGS. 7A and 7B.

In this example, signals are acquired from the sensors at step 700 with these optionally undergoing preliminary processing at step 705. The preliminary processing may be of any appropriate form, depending on the nature of the signals generated by the sensors. For example, this could include digitization of analogue sensor signals achieved using a dedicated analogue to digital convertor, and optionally filtering, noise cleaning, windowing, amplification, convolutions, or the like. This can also include performing a frequency transformation, such as a fast Fourier transform, to convert time series information into the frequency domain, which can reduce the volume of data, whilst retaining the necessary meaningful data for analysis. These can be achieved utilizing known techniques and will not be described in further detail.

At step 710 the monitoring device processor 311 generates sensor data. The sensor data can be of any appropriate form but typically includes information derived from the sensor signals. In one example, the sensor data is in the form of data packets including a data packet header and a payload indicative of the sensor signals. The payload data can include raw digitized sensor signals or values derived therefrom, such as parameters, frequency domain information, features extracted from sensor signals, or the like. Typically only limited processing is performed on board the monitoring device is simple in order to reduce processing and power requirements, but it will be appreciated that this may not always be the case and indeed additional analysis may be performed in order to reduce the amount of sensor data that needs to be transmitted to the client device 360. The data packets may also include other relevant information, such as a time or date of capture of the data. The sensor data, and in particular the raw sensor signals are also typically stored in the memory 314, for subsequent retrieval by the dock 340.

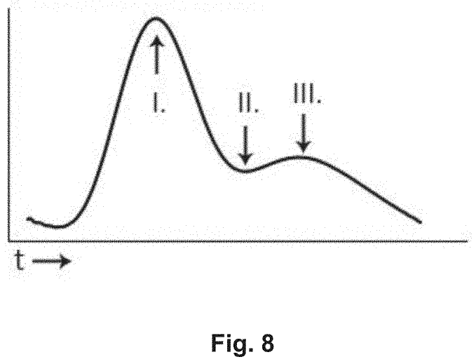

At step 715 sensor data is transmitted to the client device 360, which analyses the data at step 720 to determine features from the sensor data at step 725. In this regard, the PPG signal for a single pulse cycle is as shown in FIG. 8.

Components included in the PPG signal include a systolic peak (I) corresponding to contraction of the heart (systole), a diastolic peak (III), which is the resting period between the heart beats (diastole), while blood is flowing and a dicrotic notch (II), which is a transient minima separating the systolic & diastolic peaks. Systole and diastole occupy, respectively, about one-third and two-thirds of the cycle of heart action. When analyzing PPG signals to identify the features outlined above, the systolic peak (I) is used. Thus, for example, the heart rate is identified by measuring the number of systolic peaks in a given time frame (typically one minute). Similarly, cardiac features, such as interbeat intervals, heart rate variability and the like, can also be calculated by examining the time between successive systolic peaks.

For breathing rate, the PPG signals are analyzed to determine blood oxygenation levels. In this regard, one of the major roles of blood is to supply oxygen to tissues throughout the body. This is achieved through the protein hemoglobin within red blood cells, which has a high affinity to oxygen. Thus, as blood passes through capillaries in the lungs, the hemoglobin in red blood cells binds to oxygen which is subsequently pumped through arteries via the heart and transported to various tissues. The term blood oxygen saturation specifically refers to the proportion of hemoglobin in the blood that is carrying oxygen, and is given by:

$$Oxygen Saturation = \frac{Hb {O}_{2}}{Hb {0}_{2} + {Hb}^{′}}$$

where Hb refers to hemoglobin not bound with oxygen and HbO2 refers to hemoglobin bound to oxygen

Arterial blood oxygen saturation is typically measured using pulse oximetry which gives a percentage estimate (SpO₂). It has been established that those with a healthy respiratory system typically exhibit SpO₂ values of 96-98% at sea level. According to the World Health Organization, hypoxia is defined as a blood oxygen saturation level of less than 94%, while a blood oxygen level of less than 90% may indicate the need for clinical action.

The SpO₂ levels are calculated indirectly, through photoplethysmography (PPG), the non-invasive measurement of light absorption (usually red and infrared) through the blood. In short, when more blood is present, less light is reflected, so given a pulsatile increase in blood volume with each heartbeat, PPG effectively measures the pulse. Depending on the level of blood oxygen saturation, the PPG measurements also experience a change in the ratio of light absorbance between the red and infrared light. Namely, the extinction coefficient of oxygenated hemoglobin with red light (≈660 nm) is lower than it is for deoxygenated hemoglobin, and the reverse is true for infrared light (≈880-940 nm). Thus, a simultaneous measurement of the absorbance of both infrared and red light allows for an estimation of blood oxygen saturation.

The ratio of absorbance of infra-red to red light within the PPG sensor changes depending on the proportion of hemoglobin that is oxygenated in the blood. This change can be quantified through the so-called ratio of ratios metric, given by:

$$R = \frac{\frac{{AC}_{red}}{{DC}_{red}}}{\frac{{AC}_{infrared}}{{DC}_{infrared}}}$$

To obtain the alternating current (AC) components within the PPG measurements, the raw signals were firstly band-pass filtered between 1 Hz and 30 Hz. Peak detection is then performed on the infrared and red AC filtered signals to find their peaks and troughs using a low-pass function. The same procedure repeated on the same signals, but scaled by −1, to find the troughs. Next, the peak values and trough values are separated and interpolated, before their absolute values are added together to give a constant estimate of the AC amplitude. The direct current (DC) components were obtained by lowpass filtering the raw signals at 0.01 Hz.

This ratio is used to derive a linear approximation to calculate the SpO₂ value as a proxy to oxygen saturation, as:

SpO₂=A−B*R

where: A and B are determined empirically as A=104 and B=17.

To measure breathing rate, variations in SpO₂ values can be used to track different oxygenation levels in the blood, allowing a breathing rate to be established.

The manner in which further features can be extracted from sensor signals are known in the art, and is described in publications, such as Bruce, P., Bruce, A. & Gedeck. P. (2020), Practical Statistics for Data Scientists, 2nd edition, O'Reilly Media, Inc., Van Gent, P. (2016a). Analyzing a Discrete Heart Rate Signal Using Python. A tech blog about fun things with Python and embedded electronics, and Camm, M. J., Malik, M., Bigger, J. and Breithardt, G., 1996, Heart rate variability—Standards of measurement, physiological interpretation, and clinical use—Task Force of the European Society of Cardiology and the North American Society of Pacing and Electrophysiology, Circulation, Vol. 93, pp. 1043-1065. These will not therefore be described in any further detail. It will also be noted that this might not be required if features have already been derived by the monitoring device.

It will be appreciated that these steps are performed periodically, and optionally substantially continuously, depending on monitoring requirements, data transmission bandwidths or the like.

At step 730, the features are applied to the model(s), allowing an indicator to be generated at step 735. The manner in which this is performed will vary depending on the preferred implementation and the nature of the models. Typically this process will result in generation of a respective indicator for each of a number of different conditions, with these being displayed to the subject via a user interface displayed on the client device 330. In addition to simply generating an indicator, an alert may also be generated. For example, if the indicator indicates the subject is at a high risk of suffering from heat exhaustion or cognitive failure, an audible alert or haptic might be generated, either by the client device 330 or the monitoring device 310. Control of this could be based on thresholds, so in the event the indicator is a numerical value, if this exceeds a threshold value, then an alert could be generated, which may optionally require a user response at step 740.

At step 745, the client device 360 generates subject data, including information such as: the sensor data, the features generated, the indicator generated, and any user response provided. The subject data is uploaded to the server 330 at step 750. This can be used for logging purposes, and/or to alert third parties, particularly in the event that adverse health states are identified.

Additionally, or using raw sensor data subsequently uploaded from the dock 340, the server 330 can analyze the subject data at step 755 and optionally update the models at step 760, in turn allowing models used by the client device to be updated at step 765. In particular, this process can be performed in order to increase the accuracy of models, either improving generic models, and/or individualizing the models for specific subjects.

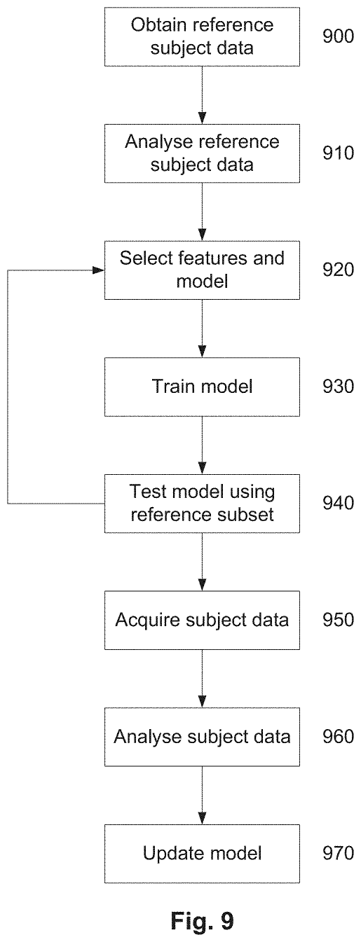

An example of the process for generating models will now be described in more detail with reference to FIG. 9.

In this example, reference subject data is obtained at step 900, which is at least partially indicative of sensor signals measured for one or more reference subjects. At step 910 the reference subject data is analyzed to determine features from the sensor signals. Steps 900 and 910 are largely analogous to steps 700, 720 and 725 described above, and it will therefore be appreciated that these can be performed in a largely similar manner, and hence will not be described in further detail.

In this example, the reference subject data is used in training a computational model, the reference subject data is also indicative of identified health states for the subject, such as whether or not the subject was suffering from, or was at risk or heat exhaustion, cognitive failure, or the like.

When using the reference subject data to train the computational model, it will be typically to determine all of the above described features, rather than just selected ones of the features, allowing this to be used in order to ascertain which of the features are most useful in discriminating between different subject health states. Nevertheless, the reference features used are as outlined above.

At step 920 a combination of the reference features and one or more generic computational models are selected, with the reference features and identified reference subject health states for a plurality of reference subjects being used to train the model(s) at step 930. The nature of the model and the training performed can be of any appropriate form and could include any one or more of decision tree learning, random forest, logistic regression, association rule learning, artificial neural networks, deep learning, inductive logic programming, support vector machines, clustering, Bayesian networks, reinforcement learning, representation learning, similarity and metric learning, genetic algorithms, rule-based machine learning, learning classifier systems, or the like. As such schemes are known, these will not be described in any further detail.

Accordingly, the above described process provides a mechanism to develop a computational model that can be used in assessing subject health state using the process described above with respect to FIG. 1.

In addition to simply generating the model, the process typically includes testing the model at step 940 to assess the discriminatory performance of the trained model. Such testing is typically performed using a subset of the reference subject data, and in particular, different reference subject data to that used to train the model, to avoid model bias. The testing is used to ensure the computational model provides sufficient discriminatory performance. In this regard, the discriminatory performance is typically based on an accuracy, sensitivity, specificity and AUROC, with a discriminatory performance of, for example 70%, being required in order for the model to be used.

It will be appreciated that if the model meets the discriminatory performance, it can then be used in determining a health state indicator using the process outlined above with respect to FIG. 1. Otherwise, the process returns to step 920 allowing different metrics and/or models to be selected, with training and testing then being repeated as required.

The above process results in a model that is generic, and in particular is not specific to any one subject. Generally, this model is initially used when new subjects are being monitored. However, as monitoring of subjects occurs, the collected subject data for that subject can be used to refine the models and individualize these to make them subject specific. Accordingly, in one example, subject data is acquired from a specific subject at step 950, with this being analyzed at step 960 and used to update the models at step 970. This is again performed by deriving features for the subject and using these, together with information regarding the health state of the subject, to retrain the models, thereby making these specific to the respective subject.

In addition to use the features to train the models, the training can also be performed taking into account reference subject attributes, so that models are specific to respective reference subject attributes or can take the subject attributes into account, such as the age, sex, ethnicity of the subject or the like. In one example, this process involves having the one or more processing devices perform clustering using the using the reference subject attributes to determine clusters of reference subjects having similar reference subject attributes, for example using a clustering technique such as k-means clustering, and then training the computational model at least in part using the reference subject clusters. For example clusters of male versus female reference individuals suffering from heat exhaustion could be identified, with this being used to train a computational model to identify the heat exhaustion in men and women respectively. It will be appreciated however that any suitable technique could be used.

In further example, the processing devices develop the model by performing one or more of feature analysis and downselection, correlation and univariate statistical separability tests and dimensionality reduction. Thus, for example, this allows for the calculation of multiple features, and multiple models, with those refined depending on their discriminatory power. Such refining can be performed using one or more of cross-validation performance, hyperparameter validation, learning curve analysis or metric relevance across models.

Accordingly, the above described techniques provide a mechanism for training one or more computational models to discriminate between different subject health states using a variety of different features, and then using the model(s) to generates indicators can assisting in identifying health states, such as heat exhaustion or the like.

Accordingly, the above described arrangement employs a predictive biometric system that utilizes large amounts of very accurate historical and real-time data which drive neural networks and machine learning approaches to produce reliable results. There are two main types of predictive biometric systems, both of which may be considered early-warning systems:
Set-Value, where predictive systems rely upon a number set by a heuristic baseline to activate an alarm. They require less compute power out of the two types, and are a required pre-cursor to stepping to a self-learning system.
Self-Learning systems which adapt to individualized thresholds and improve the more an individual uses them.

Many different types of biometric data may be used to create these systems, with the above described arrangements relying primarily on PPG readings (photo plethysmography: which measures the amount of blood in tissue) from the ear to allow for high levels of accuracy in a form-factor which is comfortable and unobtrusive for the user.

The approach can pair the data science of predictive biometric systems with medical-grade hardware to create predictive devices that can be used in dangerous real-life situations and anticipate serious medical incidents before they happen.

The above described arrangements can be used in a wide range of different applications, but are particularly useful where users are exposed to harsh or extreme conditions and where repetitive tasks are a part of their day to day role. The above arrangement is suitable for use within these environments addressing the main causes of injury or death within the heavy industries; cognitive fatigue, heat exhaustion and man down scenarios.

The above described system can employ artificial intelligence to create a unique personalized model for each user. Throughout use, base biometric data models are refined to create bespoke models for each user, increasing predictive alert accuracy. This allows for broader applications as the final model is trained to a user's unique biometric signature. Models are continuously optimized; the more often a device is worn, the more accurate are the Predictions and the more trusted the result.

The earpiece monitoring device has been designed to fit comfortably and unobtrusively behind the ear which makes it simple to use and easy to wear for hours on end. For example, the earpiece can incorporate three principles, including fluidity, durability and smoothness. Modular, malleable, removable fasteners which allow the device to sit comfortably behind the ear of each user. This design allows maximal, adjustable comfort for any wearer.

The monitoring device can implement sensing technology utilized in emergency & intensive care units across the globe, miniaturizing this approach for use on the ear ensures our readings are taken soon after blood leaves the heart and remain accurate even in times of serious distress.

The system can provide up to the minute safety risk alerts for multiple issues, and additionally can function predictively, alerting subjects that heat exhaustion or cognitive failure is likely to occur if no action is taken.

The use of a local client device allows the system to be optimized for low-connectivity areas:

On board processing allows the device to function with and without an internet connection.

Haptic early alarms on the device work without an internet connection.

The system is non-invasive, and can collect vital signs readings from the earlobe, not the ear-canal; so it can be used with earphones, headphones, or ear protection, whilst the rotating ear clip joint allows use of the device on either ears.

The predictive platform can be integrated with existing systems and equipment is key to providing the best long-term solution. From simplifying historical analysis tools all the way to incorporating real-time risk alerts with existing dashboards.

Throughout this specification and claims which follow, unless the context requires otherwise, the word “comprise”, and variations such as “comprises” or “comprising”, will be understood to imply the inclusion of a health stated integer or group of integers or steps but not the exclusion of any other integer or group of integers. As used herein and unless otherwise health stated, the term “approximately” means ±20%.

Persons skilled in the art will appreciate that numerous variations and modifications will become apparent. All such variations and modifications which become apparent to persons skilled in the art, should be considered to fall within the spirit and scope that the invention broadly appearing before described.

1) A monitoring system for monitoring a biological subject, the monitoring system including:
a) a monitoring device including:
i) a housing configured to be attached to or supported by an ear of the subject in use;
ii) one or more sensors, the one or more sensors including a photoplethysmogram (PPG) sensor provided in the housing and configured to measure attributes of blood flow within the ear;
iii) a monitoring device processor configured to:
(1) acquire sensors signals from the one or more sensors; and,
(2) generate sensor data at least partially in accordance with signals from the one or more sensors;
iv) a transmitter configured to transmit the sensor data; and,
b) one or more processing systems configured to:
i) receive the sensor data;
ii) analyze the sensor data; and, iii) generate a health state indicator indicative of a health state of the subject.

2) A monitoring system according to claim 1, wherein the PPG sensor is a transmissive PPG sensor that includes:
a) at least one radiation source provided in the housing so as to expose the ear lobe to electromagnetic radiation; and,
b) at least one radiation sensor provided in the housing so as to receive electromagnetic radiation at least one of transmitted through the ear lobe.

3) (canceled)

4) A monitoring system according to claim 1, wherein the housing includes:
a) an elongate curved main body configured to sit behind a helix of the ear; and,
b) an ear lobe clamp extending from a lower end of the main body, the ear lobe clamp being configured to receive an ear lobe of the ear so that the ear lobe is positioned between the main body and the ear lobe clamp.

5) A monitoring system according to claim 4, wherein at least one of:
a) the ear lobe clamp is rotatably mounted to the main body to allow the monitoring device to be worn on a left or right ear;
b) at least one radiation source is provided in the ear lobe clamp and wherein at least one radiation sensor is provided in the main body facing the ear lobe clamp; and,
c) the PPG sensor includes a first radiation sensor on a first side of the housing and second radiation sensor on a second side the housing and wherein the monitoring device processor is configured to:
i) monitor signals from the first and second radiation sensors;
ii) determine an active radiation sensor based on the monitored signals; and
iii) generate sensor data using signals from the active radiation sensor.

6) (canceled)

7) (canceled)

8) A monitoring system according to claim 1, wherein the housing includes a securing mechanism to secure the housing to the ear, the securing mechanism including at least one of:
a) a hook configured to extend over the ear; and,
b) a clip configured to engage an antihelix of the ear.

9) (canceled)

10) A monitoring system according to claim 1, wherein the one or more sensors include at least one of:
a) an ambient temperature sensor;
b) a skin temperature sensor;
c) a pressure sensor;
d) a humidity sensor;
e) a movement sensor;
f) an accelerometer;
g) a gyroscope;
h) an optical sensor; and,
i) a user input button.

11) A monitoring system according to claim 1, wherein the sensor data at least one of:
a) is indicative of at least one of:
i) an amount of red light transmitted through the skin;
ii) an amount of green light transmitted through the skin;
iii) an amount of infrared light transmitted through the skin;
iv) an amount of light absorbed by the skin;
v) an ambient temperature;
vi) a barometric pressure;
vii) a relative humidity;
viii) a wet bulb temperature;
ix) a skin temperature;
x) accelerometer readings; and,
xi) gyroscope readings; and,
xii) user inputs; and,
b) includes at least one of:
i) raw sensor signals; and,
ii) one or more features derived from raw sensor signals.

12) A monitoring system according to claim 1, wherein the monitoring device includes at least one of:
a) a haptic motor;
b) an optical indicator; and,
c) a speaker.

13) (canceled)

14) A monitoring system according to claim 1, wherein the monitoring device at least partially processes the sensor signals by at least one:
a) filtering;
b) amplifying;
c) digitizing; and,
d) parameterizing.

15) A monitoring system according to claim 1, wherein the system includes a dock configured to:
a) charge a power supply in the monitoring device; and,
b) retrieve sensor data stored in the monitoring device, the retrieved sensor data being transferred to one or more processing systems for analysis.

16) A monitoring system according to claim 1, wherein the one or more processing systems include a client device configured to:
a) analyze the sensor data;
b) generate a health state indicator indicative of a health state of the subject; and,
c) at least one of:
i) generate an alert;
ii) output an indication of the health state indicator; and,
iii) cause the monitoring device to generate an alert; and,
d) transfer subject data to a processing system, the subject data being indicative of at least one of:
i) sensor data;
ii) a health state indicator; and
iii) user input provided in response to output of the health state indicator.

17) (canceled)

18) A monitoring system according to claim 1, wherein the one or more processing systems include a client device configured to:
a) receive the sensor data;
b) transfer subject data to a processing system, the subject data being at least partially indicative of the sensor data;
c) receive an indication of the health state indicator from the processing system; and,
d) output the health state indicator.

19) A monitoring system according to claim 1, wherein the one or more processing systems are configured to:
a) determine one or more features derived from sensor signals;
b) use the features and at least one computational model to determine a health state indicator indicative of a subject health state.

20) A monitoring system according to claim 19, wherein the one or more processing systems are configured to:
a) compare one or more feature values to corresponding reference feature values, including at least one of:
i) baseline feature values for the subject;
ii) previous feature values for the subject;
iii) feature values derived from reference subjects having a known health state; and,
iv threshold values; and,
b) determine the health state in accordance with results of the comparison.

21) (canceled)

22) A monitoring system according to claim 19, wherein the one or more features include at least one of:
a) values of raw sensor signals;
b) a pulse feature;
c) a heart rate;
d) a mean heart rate;
e) a heart rate variability feature;
f) a breathing rate;
g) a mean breathing rate;
h) an interbeat interval of the heart rate;
i) a mean interbeat interval of the heart rate;
j) a median interbeat interval of the heart rate;
k) a standard deviation of the interbeat interval of the heart rate;
l) a median absolute deviation of the interbeat interval;
m) a standard deviation of the difference in the interbeat interval;
n) a median absolute deviation of the difference in the interbeat interval;
o) a percentage difference in Interbeat interval >50 ms;
p) a percentage difference in Interbeat interval >20 ms;
q) a square root of the mean of the successive differences between heart rates;
r) an area under the curve of the heart rate wave;
s) an energy of the power of the Heart Rate Variability (HRV) signal;
t) a proportion of the HRV energy in the Low Frequency band;
u) a proportion of the HRV energy in the High Frequency band;
v) a ratio between HRV signal within the low and high frequency bands;
w) an entropy of the HRV signal;
x) an entropy of the PPG signal;
y) a positive/negative ratio of the Systolic wave;
z) a ratio of the positive Systolic and Diastolic waves;
aa) a maximum slope of the Systolic wave;
bb) a time to peak of the Systolic wave;
cc) an energy of the PPG signal in volts;
dd) a proportion of the PPG energy in a Very Low Frequency band;
ee) a proportion of the PPG energy in a Low Frequency band;
ff) a proportion of the PPG energy in a Medium Frequency band;
gg) a proportion of the PPG energy in a High Frequency band;
hh) a ratio between the proportion of the PPG energy in the Low Frequency band and proportion of the PPG energy in the high Frequency band;
ii) a saturation of Peripheral Oxygen in the blood;
jj) a median change in accelerometer signals;
kk) a 90th quantile of the accelerometer changes;
ll) a 95th quantile of the accelerometer changes;
mm) a 99th quantile of the accelerometer changes;
nn) a maximum accelerometer change;
oo) a median change in the gyroscope signals;
pp) a 90th quantile of the gyroscope changes;
qq) a 95th quantile of the gyroscope changes;
rr) a 99th quantile of the gyroscope changes;
ss) a maximum gyroscope change;
tt) a power spectral density of Interbeat intervals;
uu) a power spectral density of Interbeat intervals in a frequency band 0.04 Hz to 0.15 Hz;
vv) a power spectral density of Interbeat intervals in a frequency band 0.16 Hz to 0.5 Hz;
ww) a ratio of power spectral density of Interbeat intervals in different frequency bands;
xx) an integral of a power spectral density of a signal;
yy) an integral of a power spectral density of a signal in a frequency band 0 Hz to 0.3 Hz;
zz) an integral of a power spectral density of a signal in a frequency band 1.2 Hz to 1.9 Hz;
aaa) a mean ambient temperature;
bbb) an ambient temperature range;
ccc) a mean wet temperature;
ddd) a wet temperature range;
eee) a wet temperature standard deviation;
fff) a mean skin temperature;
ggg) a skin temperature standard deviation;
hhh) a skin temperature range;
iii) a mean ambient relative humidity;
jjj) an ambient relative humidity standard deviation;
kkk) an ambient relative humidity range;
lll) a mean ambient pressure;
mmm) an ambient pressure standard deviation;
nnn) an ambient pressure range;
ooo) a PPG vector saturation;
ppp) a PPG vector noise;
qqq) a PPG vector noise scale; and,
rrr) a PPG vector signal variance.

23) (canceled)

24) A monitoring system according to claim 19, wherein the one or more processing systems are configured to use the one or more features and a computational model to determine the health state, the at least one computational model being at least partially indicative of a relationship between different subject health states and one or more features, wherein at least one of:
a) the computational model is optionally obtained by one of:
i) applying machine learning to reference features derived from one or more reference subject having known health states and applying machine learning to features derived from the subject; and,
ii) developing a generic model by applying machine learning to reference features derived from one or more reference subjects having known health states and modifying a generic model to create a subject specific model by applying machine learning to features derived from the subject; and,
b) the at least one computational model includes at least one of:
i) one or more respective computational models for each of a plurality of health states;
ii) boosted classifiers that classify aggregated time segments into categories relating to at least one health state;
iii) a rolling auto-regressive integrated moving average model applied to key features and raw data to predict risk of at least one health state; and,
iv) a long short-term memory model using a recursive deep learning approach which utilizes a previous hours' worth of data to predict risk of at least one health state.

25) (canceled)

26) (canceled)

27) (canceled)

28) (canceled)

29) A monitoring system according to claim 1, wherein the health state indicator is indicative of one or more of a plurality of health states, including at least one of:
a) cognitive fatigue;
b) heat stress;
c) a risk of cognitive fatigue;
d) a risk of heat stress; and,
e) collapse or non-responsiveness.

30) A monitoring system according to claim 1, wherein at least one of:
a) a risk of cognitive fatigue is determined using at least one of:
i) a low frequency band of the heart rate signal;
ii) a high frequency band of the heart rate signal;
iii) a power of the heart rate signal;
iv) a barometric pressure;
v) a humidity;
vi) an ambient temperature;
vii) a PPG signal;
viii) an entropy of the PPG signal;
ix) a mean interbeat interval of the heart rate;
x) a median interbeat interval of the heart rate; and,
b) a risk of heat stress is determined using at least one of:
i) raw PPG signals;
ii) a skin temperature;
iii) a wet bulb temperature;
iv) an ambient temperature;
v) a relative humidity;
vi) a barometric pressure;
vii) an entropy of a heart rate variability signal;
viii) a square root of the mean of the successive differences between heart rates;
ix) a mean interbeat interval of the heart rate;
x) a median interbeat interval of the heart rate;
xi) UV exposure levels; and,
xii) an area under the curve of the Heart Rate wave.
c) a collapse or non-responsiveness is determined using at least one of:
i) a heart rate;
ii) a change in heart rate;
iii) a PPG signal;
iv) a change in blood oxygenation;
v) accelerometer readings;
vi) gyroscope readings;
vii) a median change in accelerometer signals;
viii) a 90th quantile of the accelerometer changes;
ix) a 95th quantile of the accelerometer changes;
x) a 99th quantile of the accelerometer changes;
xi) a maximum accelerometer change;
xii) a median change in the gyroscope signals;
xiii) a 90th quantile of the gyroscope changes;
xiv) a 95th quantile of the gyroscope changes;
xv) a 99th quantile of the gyroscope changes; and,
xvi) a maximum gyroscope change.

31) (canceled)

32) (canceled)

33) A method system for monitoring a biological subject including:
a) using a monitoring device including:
i) a housing configured to be attached to or supported by an ear of the subject in use;
ii) one or more sensors, the one or more sensors including a photoplethysmogram (PPG) sensor provided in the housing and configured to measure attributes of blood flow within the ear; and,
iii) a monitoring device processor to:
(1) acquire sensors signals from the one or more sensors; and,
(2) generate sensor data at least partially in accordance with signals from the one or more sensors;
b) using a transmitter to transmits the sensor data; and,
c) using one or more processing systems to:
i) receive the sensor data;
ii) analyze the sensor data; and,
iii) generate a health state indicator indicative of a health state of the subject.

34) (canceled)

35) (canceled)

36) A monitoring device for monitoring a biological subject, the monitoring device including:
a) a housing configured to be attached to or supported by an ear of the subject in use, wherein the housing includes:
i) an elongate curved main body configured to sit behind a helix of the ear; and,
ii) an ear lobe clamp extending from a lower end of the main body, the ear lobe clamp being configured to receive an ear lobe of the ear so that the ear lobe is positioned between the main body and the ear lobe clamp and the ear lobe clamp being rotatably mounted to the main body to allow the monitoring device to be worn on a left or right ear;
b) one or more sensors, the one or more sensors including a photoplethysmogram (PPG) sensor provided in the housing and configured to measure attributes of blood flow within the ear, wherein the PPG includes at least one radiation source is provided in the ear lobe clamp and wherein at least one radiation sensor is provided in the main body facing the ear lobe clamp; and,
c) a monitoring device processor configured to:
i) acquire sensors signals from the one or more sensors; and,
ii) generate sensor data at least partially in accordance with signals from the one or more sensors.
